(19) United States
(12) Patent Application Publication
(10) Pub. No.: US 2013/0190628 A1
(43) Pub. Date: Jul. 25, 2013

(54) WIRE-TYPE WAVEGUIDE FOR TERAHERTZ RADIATION

(71) Applicants: Lorenzo Tripodi, Eindhoven (NL); Jaime Gomez Rivas, Eindhoven (NL); Peter Gerard Van Leuven, Venlo (NL); Martijn Constant Van Beurden, Eindhoven (NL); Audrey Anne-Marie Berrier, Eindhoven (NL); Marion Kornelia Matters-Kammerer, Valkenswaard (NL)

(72) Inventors: Lorenzo Tripodi, Eindhoven (NL); Jaime Gomez Rivas, Eindhoven (NL); Peter Gerard Van Leuven, Venlo (NL); Martijn Constant Van Beurden, Eindhoven (NL); Audrey Anne-Marie Berrier, Eindhoven (NL); Marion Kornelia Matters-Kammerer, Valkenswaard (NL)

(73) Assignee: KONINKLIJKE PHILIPS ELECTRONICS N.V., EINDHOVEN (NL)

(21) Appl. No.: 13/877,670

(22) PCT Filed: Oct. 5, 2011
(86) PCT No.: PCT/IB11/54378

§ 371 Date: Apr. 4, 2013

(30) Foreign Application Priority Data
Oct. 12, 2010 (EP) 10187312.3

(51) Int. Cl. H01P 3/10; A61B 5/00; A61B 1/06; G01J 5/08

(52) U.S. Cl. CPC H01P 3/10; G01J 5/0818; A61B 5/0075; A61B 1/06
USPC 600/476; 333/239; 333/1; 250/338.1; 600/104

(57) ABSTRACT

In order to guide electromagnetic waves in the terahertz range over long distances of several meters with low bending losses and large bandwidth, a device, a system and a method are provided such that electromagnetic waves in the terahertz range can be coupled into a wire having a core structure and at least one confinement structure, wherein the confinement structure extends continuously along a length of the wire.

### FIELD OF THE INVENTION

The present invention relates to a device, a system and a method for guiding electromagnetic waves in the terahertz range.

### BACKGROUND OF THE INVENTION

Free-space propagation of electromagnetic radiation is widely used in modern technology. Common applications are, for instance, satellite communications, broadcasting of television signals and radar. In many cases, though, guided propagation of waves is indispensible. Examples are long-haul fiber optics communications and coaxial cable guiding of TV signals. Guided propagation of optical (visible and infrared) or microwave signals is a problem solved long ago with the invention of the optical fiber and the microwave waveguide, wherein waves are confined to propagation in one dimension. Commercial coaxial cables are able to carry radiation up to 67 GHz and for higher frequencies rectangular metallic waveguides are considered suitable, when the bandwidth of the signal is relatively narrow.

In the past 30 years, terahertz (THz) radiation has attracted the interest of the scientific and engineering community for its wide range of possible scientific and commercial applications. Terahertz radiation relates to electromagnetic waves in the terahertz range, i.e. between 100 GHz and 3 THz, also referred to as sub-millimeter radiation. The terahertz band is located between the high-frequency edge of the microwave range and the long-wavelength edge of far-infrared light. The facts that the vibrational modes of several molecules lie in this part of the spectrum and that water very easily blocks waves at those frequencies, make terahertz radiation a suitable probe to investigate spectroscopic properties of materials, which are usually inaccessible with infrared, X-rays, or other types of probe signals. Moreover, terahertz radiation is non-ionizing and thus not expected to damage tissues and DNA. Since some frequencies can penetrate several millimeters of tissue and are then reflected back, terahertz radiation is also used for medical imaging. However, although the potential and uniqueness of this type of radiation are quite evident nowadays, commercial terahertz imaging and spectroscopy systems are still not very common on the market. One of the major reasons for this is the intrinsic technological difficulty of producing, detecting, and in particular guiding terahertz radiation with devices at a reasonable cost for market applications. Moreover, the bandwidth of terahertz signals commonly required in terahertz imaging and spectroscopy is extremely broad. Thus, a waveguide for terahertz radiation must be suited for a large bandwidth.

Recently, parallel-plate waveguides have been proposed that are adapted to guide sub-picosecond terahertz pulses. However, this type of waveguide cannot be easily manufactured for spectroscopic or imaging applications, because the beam can travel in one direction only and its distortion becomes quite large for lengths above few centimeters. Moreover, in this type of waveguide, one dimension remains unguided, which leads to diffraction of the beam and relevant losses for longer lengths. As an improvement of the parallel-plate waveguide, a combination of a parallel-plate waveguide and metallic posts fabricated using standard MEMS technology has been suggested. Even if the bandwidth achieved using micro-fabrication techniques is relatively large (e.g. in the order of 0.5 THz), this waveguide is still affected by the problem of cut-off frequencies, as it is typical for rectangular waveguides. Moreover, this waveguide remains relatively expensive and inconvenient for other applications requiring propagation lengths in the order of several meters. Also, such parallel-plate waveguides are strongly limited in their applications due to their geometric dimensions and low flexibility. In a new approach described in the article “Metal wires for terahertz wave-guiding” by Kanglin Wang and Daniel M. Mittleman in Letters to Nature, terahertz waves are guided by means of a metal wire. However, the problem of this device is that its guiding capability is very limited and when bending the wire with low bending radius, the guided field easily escapes into the air, which leads to a limitation of practical applications due to high bending losses. Moreover, the radiation is not confined to the inside of the wire but remains concentrated at its surface and could, e.g. in an endoscopic application in the human body, easily interact with parts of the body not concerned by the analysis.

### SUMMARY OF THE INVENTION

In view of above disadvantages and problems in the prior art, it is an object of the present invention to provide a device, a system and a method for guiding electromagnetic waves in the terahertz range, wherein a wide bandwidth, a long propagation length and low bending losses are achievable at reasonable costs and production effort.

The object is solved by the features of the independent claims.

The invention is based on the idea to confine a propagating electromagnetic field of terahertz radiation, i.e. with frequencies from below 100 GHz up to several terahertz, in a space with sub-wavelength dimensions. This is achieved by using a wire having a cross-section with sub-wavelength structures that are smaller than the smallest wavelength of the guided radiation. For instance, terahertz radiation with a frequency of 100 GHz corresponding to the longest wavelength of terahertz radiation has a wavelength in free space of 1 mm. Thus, the wire should comprise a structure smaller than 1 mm.

In one aspect of the present invention, a device is provided for guiding electromagnetic waves in the terahertz range comprising a wire. The wire includes a core structure and at least one confinement structure, wherein the confinement structure extends continuously along a longitudinal direction of the wire. The confinement structure refers to a structure on a surface of the core structure, by which terahertz radiation can be confined. Since the confinement structure extends continuously along the length of the wire, the cross-sectional shape of the wire remains constant at any point along the length of the wire. For instance, industrially manufactured profiled wires can be used in order to reduce production costs of the device. By these means, terahertz waves can be guided with negligible losses over distances of a few meters.

In a preferred embodiment, the confinement structure includes at least one groove or rib. In case that the confinement structure is designed as a groove, an insertion or depression is formed in the core structure of the wire. If the confinement structure is designed as a rib, a protrusion or bulge is formed along the wire prominent from the core structure. Preferably, the confinement structure has an angled cross-sectional shape, e.g. a substantially triangular, rectangular and/or poly-angular cross-section. Possibly, the confinement structure is composed of at least one groove and at least one rib, thus resembling for instance at a N-shape or a W-shape.

Furthermore, the core structure may have a substantially circular cross-section. That is, the core structure has a circular cross-section except for a portion, where the confinement structure is located, i.e. except for a cut-out portion in case of a groove or a bulge portion in case of a rib. Alternatively, the core structure may have a substantially triangular, rectangular, poly-angular or star-like cross-section. In case of a triangular core structure, the confinement structure may consist in the vertices of the triangle. Likewise, in case of a star-like core structure with many cusps, the indentations may function as groove-type confinement structures and/or the cusps may function as rib-type confinement structures. Moreover, the cross-section of the core structure and/or of the wire may be asymmetric.

Preferably, the confinement structure has at least one dimension with sub-wavelength dimension. Hence, the cross-section of the confinement structure has at least one portion, which is smaller than a wavelength of the guided electromagnetic waves. In case that a large bandwidth of electromagnetic waves is guided, the confinement structure may have at least one dimension smaller than the smallest wavelength of the bandwidth. Preferably, dimensions of the confinement structure in the cross-section are smaller than the diameter of the core structure.

At least one of the core structure and the confinement structure may be made of a conducting material and/or a semi-conductor material. If the core structure and/or the confinement structure is made of a conducting material, this may include any metal, preferably copper or stainless steel. When using a semi-conductor for at least one of the core structure and the confinement structure, the electrical characteristics of the wire may be adjusted using doping agents. Possibly, the core structure and the confinement structure are made of the same material. By using common easily-processible materials such as copper, manufacturing costs can be reduced.

In a preferred embodiment, the wire is flexible. Hence, the wire may be designed such that it can be bent with a small bending radius. Thus, it may be used to guide terahertz waves to examination areas that are difficult to access, for instance, when applied in a terahertz endoscope or catheter.

Furthermore, the wire may be designed such that the electromagnetic waves propagating along the wire have at least one propagation mode substantially confined within the confinement structure and/or within a cross-section of the wire. For instance, in case of a groove-type confinement structure having a V-shape, a propagation mode of the guided electromagnetic waves may be confined in the bottom of the V-shape. Then, it is also confined within the cross-section of the wire. By these means, bending losses can be reduced as well as unwanted interactions with the environment surrounding the wire. Thus, the device is suited for endoscopic applications in the human body.

The wire may additionally comprise a coating, for instance a low-loss coating reducing radiation losses when guiding the terahertz waves to an area of interest. Possible materials for the coating include benzocyclobuten, polystyrene, polyethylene and any other low-loss dielectric or a combination thereof. This will also lead to a better confinement of the guided radiation. Alternatively, the coating may be made of a metal. By these means, the coating can prevent electromagnetic waves guided by the wire to interact with material outside the wire. Hence, unintended exposure to terahertz radiation along the wire can be avoided. Also, energy losses of the guided terahertz radiation and in particular bending losses can be reduced, resulting in increased propagation lengths.

Advantageously, the coating may form the outer surface of the wire. Thus, it surrounds the core structure as well as the confinement structure. For instance, if the confinement structure is a groove, the coating may fill the groove. On the other hand, if the confinement structure is a rib-type confinement structure, the coating may enclose the protruding rib-type confinement structure as well as the core structure. Preferably, the coating results in a uniform surface of the wire. For instance, the wire including the coating may have a circular, triangular or rectangular cross-sectional shape. By these means, the coating can prevent the accumulation of material at corners or in grooves on the wire surface, thus avoiding a contamination of the wire.

The wire may comprise more than one confinement structure. For instance, two confinement structures may extend along the wire on opposite sides of the wire. Possibly, the two confinement structures are designed such that the propagation fields of electromagnetic waves traveling along the wire are coupled to each other. Moreover, the wire may comprise at least two confinement structures, wherein at least one confinement structure is adapted to function as a transmitting channel for sending terahertz waves from a terahertz source to an examination area and at least one other confinement structure is adapted to function as a receiving channel to transmit electromagnetic waves from the examination area to a detection unit. In particular, with four confinement structures, two separate propagation modes are present that can be used independently, for instance for transmitting and receiving signals at the same time.

In another example, the wire may comprise four confinement structures, e.g. spaced apart from each other by an angle of approximately 90°. Here, two of the confinement structures that are facing each other may form a pair of confinement structures, wherein electromagnetic waves guided along these confinement structures are coupled to each other. In this case, one pair of confinement structures may be used as a transmitting channel for transmitting terahertz waves towards an area of interest, whereas the other pair of confinement structures may be used as a receiving channel for receiving electromagnetic waves reflected from the area of interest. This is in particular useful for terahertz imaging, e.g. in spectroscopic or endoscopic applications in reflection mode.

For medical interventions, the device may further comprise a needle and/or a catheter, wherein the wire is arranged in a central hole of the needle and/or the catheter. By these means, a terahertz endoscope or terahertz catheter can be realized. In addition, an output director, such as a mirror, may be provided at one end of the wire.

Furthermore, the device may be designed such that it can be applied in an endoscopic system for medical imaging, in a terahertz spectroscopic system and/or in a probe station using integrated circuits. By using sub-wavelength field confinement, the spatial resolution of the imaging and spectroscopy systems can be increased. In addition, longer propagation lengths can be achieved, so that longer distances between a terahertz source and an examination area become possible.

In another aspect of the present invention, a system for terahertz imaging is provided comprising a terahertz signal generator, a terahertz signal detector and a device according to one of the preceding claims. The system may further comprise at least one coupling unit for coupling electromagnetic waves in the terahertz range into the wire of the device. Possibly, the same or an additional coupling unit is used for coupling electromagnetic waves coming from the area of interest into the terahertz signal detector. Moreover, filter units, signal processors, display units, memories and the like may be provided. Such a system may be applied in medical imaging systems, e.g. for endoscopic applications inside the human body. The system may also be applicable in terahertz imaging systems, e.g. for analyzing material, or for high-frequency measurements of integrated circuits using probe stations.

In a further aspect of the present invention, a method is provided for guiding electromagnetic waves in the terahertz range. For this, electromagnetic waves in the terahertz range are coupled into a wire having a core structure and at least one confinement structure extending in a longitudinal direction of the wire. By means of the wire, which can be designed according to any embodiment described above, the electromagnetic waves can be guided towards an area of interest with acceptable losses.

### BRIEF DESCRIPTION OF THE DRAWINGS

In the figures:

FIG. 1A shows a sectional view illustrating a wire according to one embodiment of the invention;

FIG. 1B shows a sectional view illustrating a wire according to another embodiment of the invention;

FIG. 2 illustrates the logarithm of the normalized z-component of the Poynting vector of a propagation mode along a wire as shown in FIG. 1A; The dark box-shaped areas are part of the figure in which the field is not calculated due to numerical difficulties.

FIG. 3 shows a graph illustrating a dependence of a propagation length on a depth of a confinement structure in a wire as shown in FIG. 1A;

FIG. 4 shows a graph illustrating a dependence of a propagation length on a frequency of electromagnetic waves guided along a wire as shown in FIG. 1A;

FIG. 5A shows a sectional view illustrating a wire according to a further embodiment of the invention;

FIG. 5B shows a graph illustrating a dependence of a propagation length on a depth of a confinement structure in a wire as shown in FIG. 5A;

FIG. 6A-6C show sectional views illustrating further embodiments of a wire according to the present invention;

FIG. 7A-7C show sectional views illustrating further embodiments of a wire according to the present invention;

FIG. 8A-8C show sectional views illustrating further embodiments of a wire according to the present invention;

FIG. 9A and 9B show sectional views illustrating embodiments of a wire according to the present invention having a coating;

FIG. 10A shows a wire according to the present invention within a needle for medical applications;

FIG. 10B shows a sectional view of the assembly shown in FIG. 9A along line A-A; and

FIG. 11 shows a system for terahertz imaging according to the present invention.

### DETAILED DESCRIPTION OF THE EMBODIMENTS

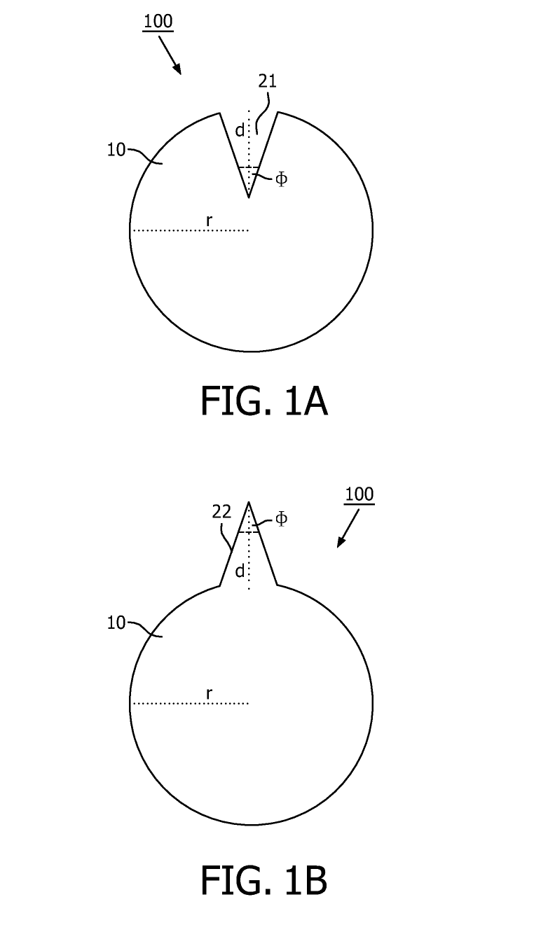

In FIG. 1, possible simple forms of a cross-section of a wire according to the present invention are shown. In FIG. 1A, a cylindrical conducting wire 100 made from copper or similar well-conducting materials is provided with a triangular V-groove 21 along its longitudinal axis. The wire 100 with radius r consists of a quasi-circular core structure 10, in which the V-shaped groove 21 is inserted with a depth of the groove d and an opening angle of the groove φ. The groove 21 extends along the whole length of the wire 100, so that the cross-section of the wire 100 remains constant over the whole length of the wire 100. In the embodiment as shown in FIG. 1A, the groove 21 functions as a confinement structure that can confine terahertz radiation. In another embodiment as shown in FIG. 1B, the confinement structure is realized as a triangular rib 22 extending along the wire 100. The rib 22 has an opening angle φ and a height d and protrudes from the circular core structure 10. These V-shaped confinement structures can be easily fabricated by dragging the wire over a sharp preform. Thus, the wires 100 may be produced at low cost by common manufacturing techniques for profiling metal wires.

Numerical simulations have been carried out to show the possibility to use the wire according to the present invention as a high-frequency waveguide. In these simulations, several propagating modes have been identified, e.g. quasi-TM₁, quasi-TM₂, V₁, V₂, etc.. The quasi-TM mode refers to a perturbation of the fundamental transverse magnetic (TM) mode in a perfectly circular wire, which is no longer strictly transverse magnetic for a non-circular wire. Likewise, the V₁ and V₂ mode refer to perturbations of the two hybrid HE₁₁ modes in a perfectly circular wire. The most interesting mode, however, is the V₁ mode, which is almost fully confined when travelling along a wire according to the present invention.

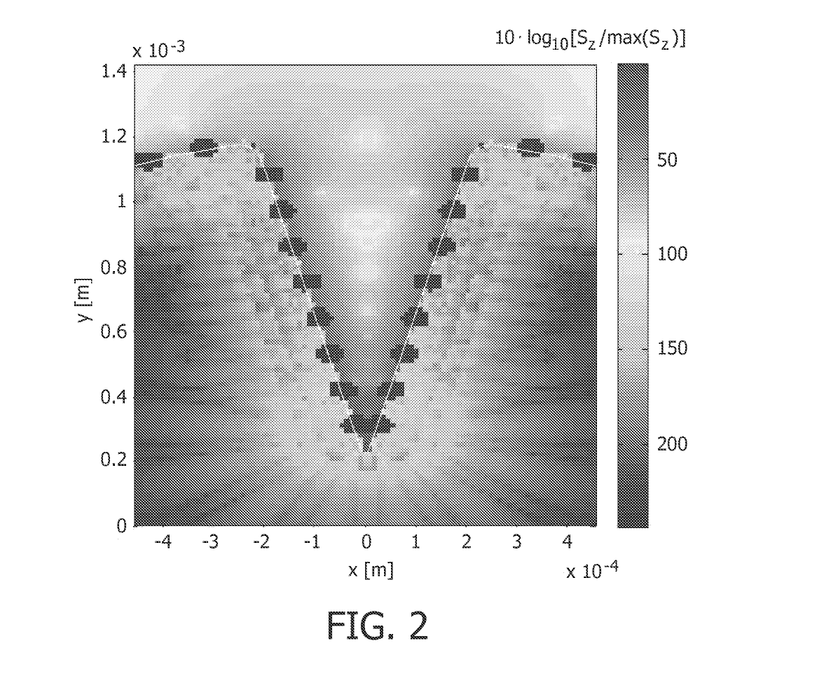

In FIG. 2, the logarithm of the normalized z-component of the Poynting vector of the V₁ mode is reported, with the z-axis being orthogonal to the plane of projection. The Poynting vector represents the energy flux of an electromagnetic field. For the simulation shown in FIG. 2, a wire 100 made of copper is used having a cross-sectional shape as shown in FIG. 1A. In this example, the frequency of the propagating signal is ƒ=300 GHz, the radius of the wire is r=1.2 mm, the depth of the groove 21 is d=1 mm and the opening angle is φ=25°. Thus, as shown in FIG. 2, the electromagnetic field of the V₁ mode is fully confined within the cross-section of the wire 100, more precisely even within the groove 21. Therefore, it is less sensitive to bending losses. Moreover, the confinement avoids the interaction between the propagating electromagnetic field and external objects surrounding the wire 100. This is in particular relevant, when employing the wire 100 in endoscopic applications inside the human body. Since the other propagation modes of the electromagnetic waves are not well confined, the interaction of the propagating electromagnetic field with external material is not avoided and bending losses are higher.

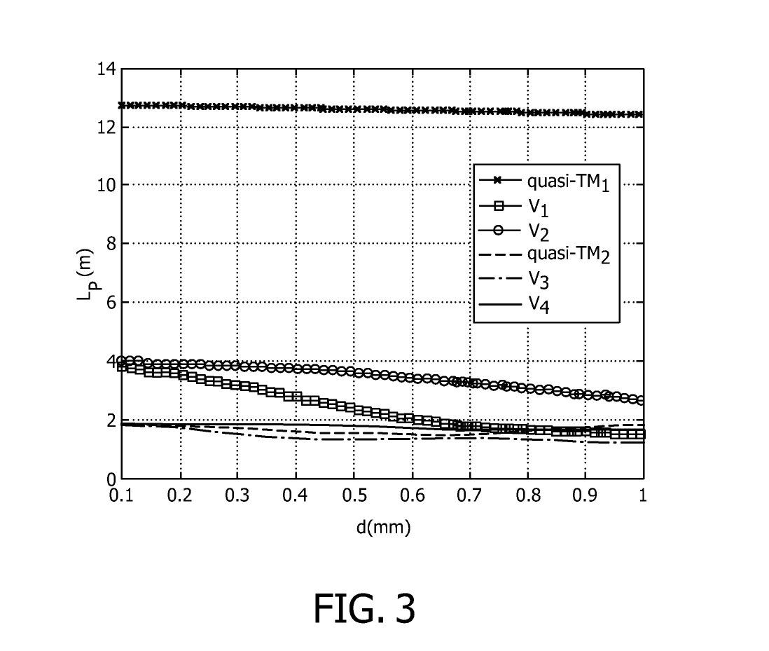

In FIG. 3, propagation lengths Lₚ for several modes are shown as a function of the depth d of the confinement structure. The propagation length is defined as the distance for electromagnetic intensity to decay by a factor of 1/e. In FIG. 3, the simulations are calculated for a copper wire 100 having a triangular groove as shown in FIG. 1A, with a wire radius r=1.2 mm, the opening angle φ=25°, the frequency of the propagating signal ƒ=300 GHz and the surrounding of the wire being air. As can be seen from FIG. 3, the propagation length Lₚ decreases with the depth of the confinement structure d. The propagation mode of interest, i.e. the V₁ mode, has a propagation length of more than 2 m for a depth d of the groove 21 lower than 0.6 mm and more than 1.5 m for a depth d of 1 mm. It is important to note that the exemplary propagating signal has a frequency of 300 GHz, which results in a wavelength of 1 mm in free space. As can be seen in FIG. 2, the signal is confined to a fraction of the groove 21, so that propagation and sub-wavelength confinement are achieved. Thus, electromagnetic waves in the terahertz range, i.e. with wavelengths in the millimeter range, can be guided with low losses for considerable distances of several meters.

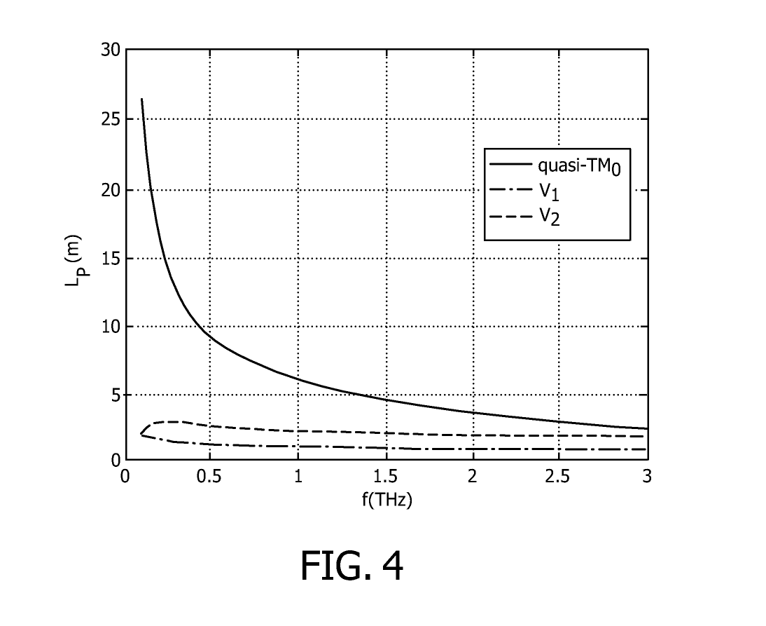

FIG. 4 shows the dependence of the propagation length Lₚ on the frequency of the guided electromagnetic signal. Here, even for a large bandwidth of frequencies, e.g. from 300 GHz to 3 THz, the propagation length Lₚ remains substantially constant for the V₁ mode. Hence, the wire-type waveguide according to the present invention is able to sustain signals with an extremely large bandwidth from 100 GHz up to several terahertz. This in particular useful for spectroscopic or imaging applications, since they require extremely large bandwidths.

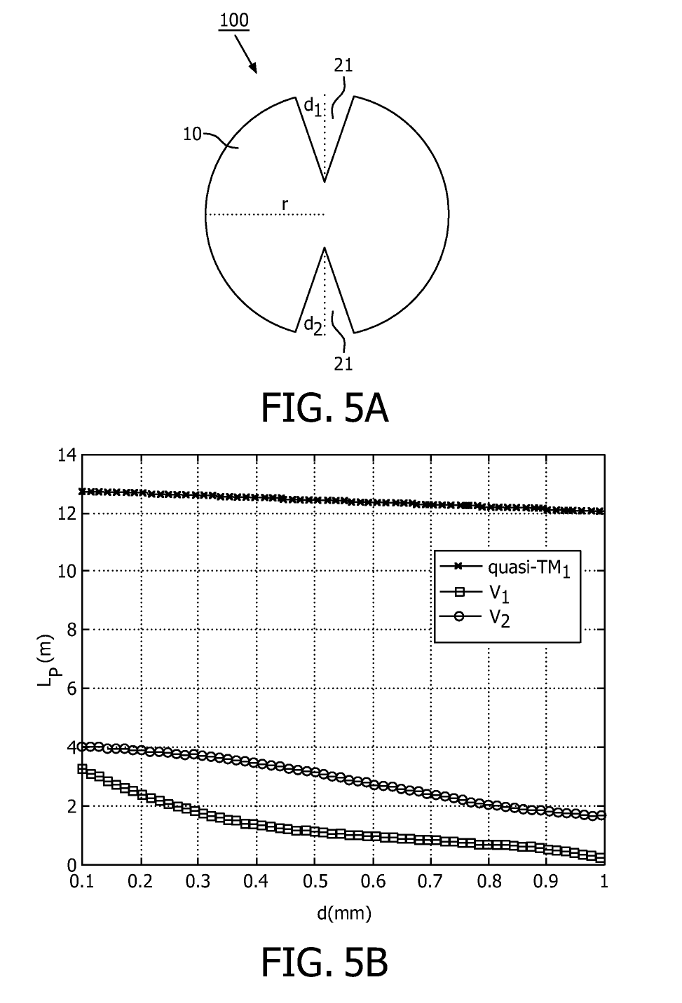

The features of the device including a wire 100 according to the present invention were explained above using the example of a wire 100 as shown in FIG. 1A. However, the present invention is not limited to this wire shape. On the contrary, also other cross-sectional shapes of the wire 100 can be used, e.g. as shown in FIG. 1B or in FIGS. 5A, 6 and 7.

In FIG. 5A, a wire 100 is shown having a quasi-circular core structure 10 and two V-shaped grooves 21. The grooves 21 may have different depths d₁ and d₂ as well as different opening angles. In a wire-type waveguide having two confinement structures on opposing sides of the wire 100, electromagnetic signals travelling along the pair of confinement structures can be coupled. In FIG. 5B, the propagation lengths Lₚ of different modes and their dependence on the depth d of the grooves 21 are shown for the double-groove wire 100. Here, the frequency of the electromagnetic signal propagating along the wire 100 is 300 GHz and the grooves 21 have equal depths (d=d₁=d₂). As can be seen, the propagation length of the V₁ mode is more than 4 m, if the groove depths d are about 0.3 mm. Hence, the guiding capability is still present in a wire 100 having two confinement structures.

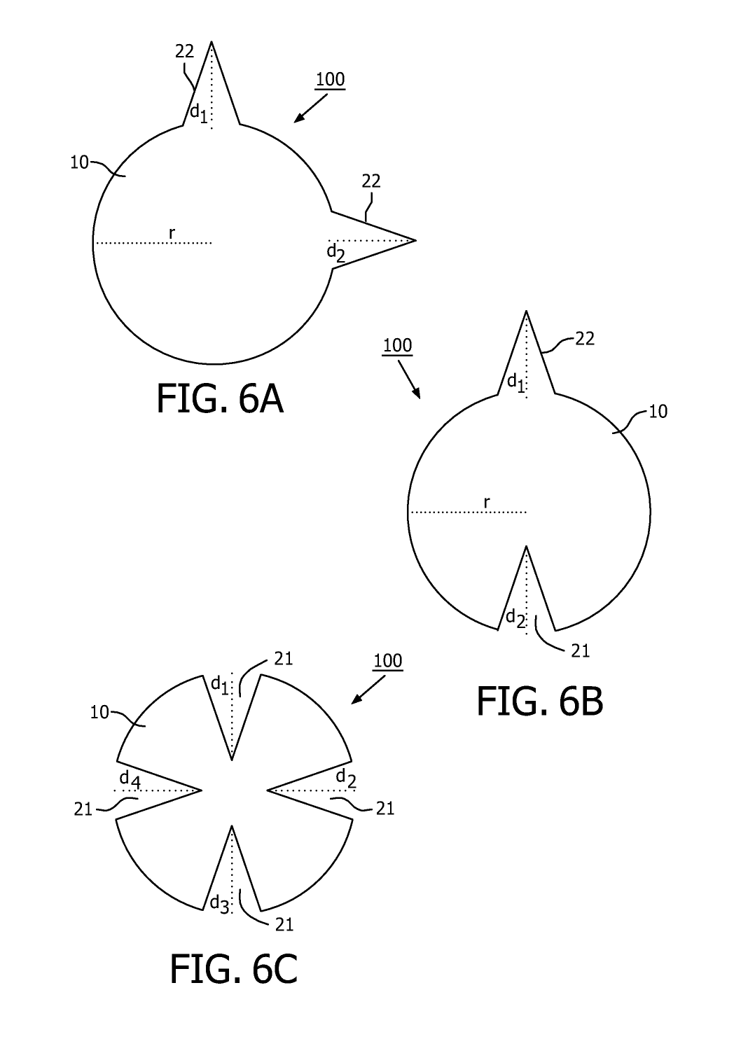

In further embodiments of the wire-type waveguide according to the present invention, the number of confinement structures, i.e. of grooves 21 and ribs 22, can be increased to two or more. Examples for cross-sectional shapes of the wire 100 are shown in FIGS. 6, 7 and 8. In FIG. 6A, a wire 100 is shown having a circular core structure 10 and two triangular ribs 22 that are spaced apart from each other by an angle of 90°. In a wire 100 having two independent confinement structures that are not coupled to each other, as for instance the wire 100 shown in FIG. 6A, the confinement structures can be used as separate transmitting and receiving channels, respectively. Hence, one channel can be used to guide a terahertz signal towards an area of interest, whereas the other channel can be used to propagate the reflected signal from the area of interest back to a signal detector. By these means, complicated multiplexing techniques can be avoided, without requiring separate waveguides for transmitting and receiving.

FIG. 6B exemplarily illustrates a wire 100 having a rib 22 as well as a groove 21 having a triangular shape. However, the rib 22 and the groove 21 can have different shapes and dimensions. Also, the present invention is not limited to this geometric arrangement of the confinement structures, but the confinement structures can be appropriately arranged at the cross-section of the core structure 10 in various ways.

FIG. 6C shows another embodiment for a wire 100 having a plurality of confinement structures. In the example shown, four grooves 21 are spaced apart with regular intervals arranged at a quasi-circular cross-section of the core structure 10 with an angle of 90° there between. As mentioned above, the confinement structures can have different sizes and shapes as well as different distances from each other. For a wire 100 having four confinement structures, two confinement structures facing each other can form a pair, respectively, and each pair of confinement structures can be used as a separate transmitting or receiving channel. For instance, one channel can be used to guide an endoscopic probe signal sent by a terahertz signal generator to a sample and the other channel can be used simultaneously to propagate the reflected signal from the sample back to a signal detector. By these means, imaging applications are possible using only a single waveguide according to the present invention.

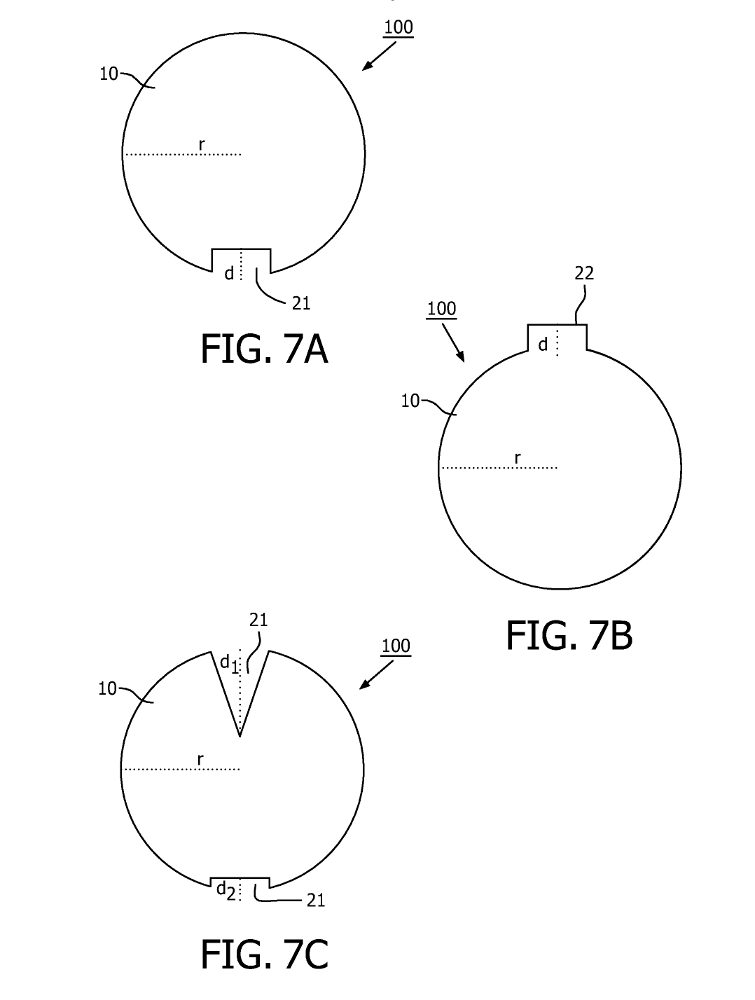

Further embodiments including a rectangular confinement structure are shown in FIG. 7A-7C. In FIG. 7A, a wire 100 has a quasi-circular core structure 10 with a rectangular groove 21 extending along the length of the wire 100, whereas the wire shown in FIG. 7B has a rectangular rib 22. In FIG. 7C, it is furthermore illustrated that rectangular and triangular confinement structures can be combined. Hence, any positive or negative confinement structures, i.e. ribs 22 or grooves 21, can be employed having triangular, rectangular or poly-angular shapes.

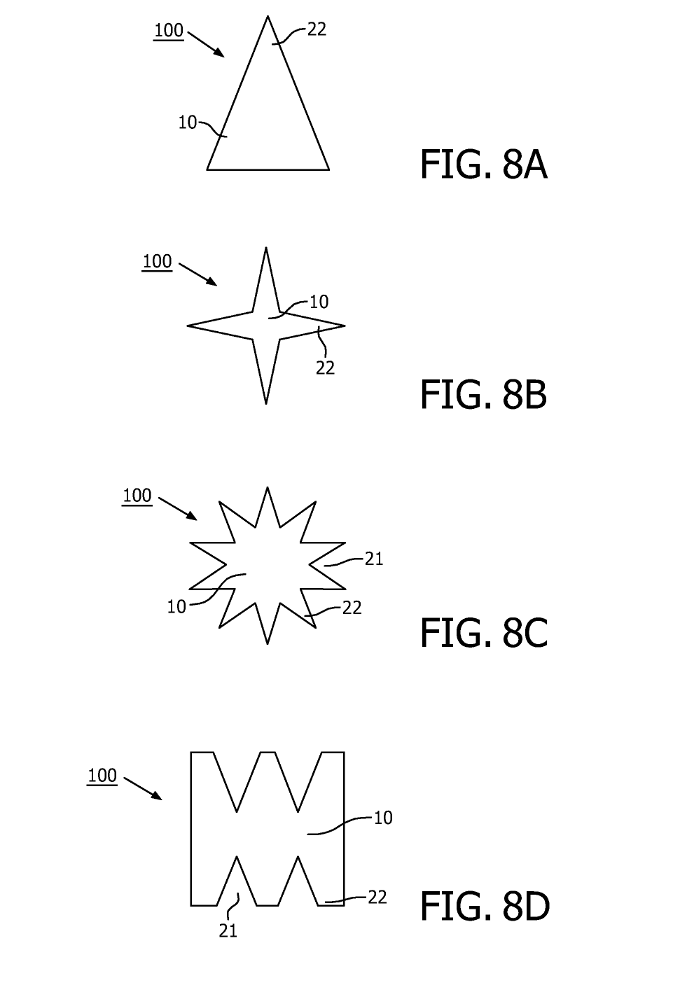

Moreover, as shown in FIG. 8, the core structure 10 of the wire 100 is not limited to a circular or quasi-circular cross-section. For instance, as shown in FIG. 8A, the core structure 10 can have a triangular cross-section, wherein the vertices of the triangle may function as positive rib-like confinement structures 22. In FIG. 8B and 8C, core structures 10 having a star-like cross-section are shown. Here, the vertices may function as rib-like confinement structures 22, whereas the indentations between the vertices may represent groove-like confinement structures 21. In a further example as shown in FIG. 8D, a core structure 10 having a rectangular cross-section is shown. Here, confinement structures such as grooves 21 and ribs 22 can be formed on the surface of the core structure 10.

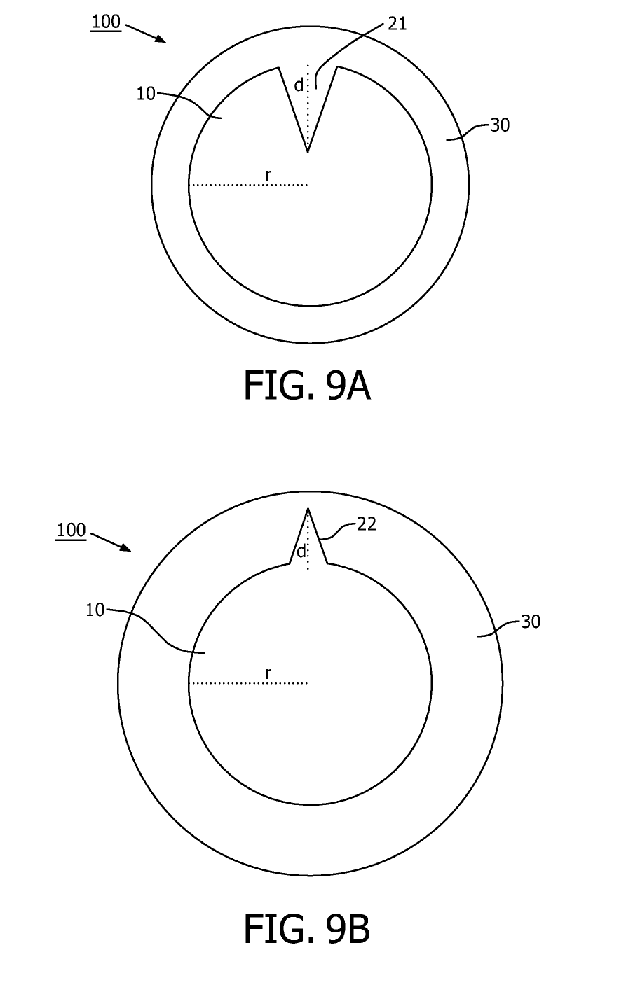

In a further embodiment of the present invention, the wire 100 may additionally comprise a coating 30, as shown in FIG. 9. In FIG. 9A, a quasi-circular core structure 10 having a V-shaped groove 21 is surrounded by the coating 30. The coating 30 fills the groove 21, thereby avoiding foreign material from accumulating in the groove, when the wire 100 is used. The coating 30 can be made of any low-loss dielectric, e.g. benzocylobutene (BCB) or cheaper materials like polyethylene or polystyrene, which reduces radiation losses and leads to a better confinement. Alternatively, the coating 30 may be made of metal or other materials suitable to reduce radiation losses under bending conditions. Instead of a conducting material, semi-conductor material can be used for the wire 100, adding an additional degree of freedom in the design of these waveguides due to the doping level. In FIG. 9B, a circular core structure 10 having a triangular rib 22 is enclosed by the coating 30. When the coating 30 surrounds the core structure 10 as well as the confinement structures of the wire 100 and thus forms the outer surface of the wire 100, radiation losses and interaction with the surroundings of the wire 100 can be reduced. Thus, for a wire 100 comprising the coating 30, the terahertz waves are almost fully confined within the cross-section of the wire 100 regardless of the confinement structure. This is particularly useful for positive confinement structures, i.e. rib-type confinement structures 22, since for these, the ability to confine the electromagnetic field inside the cross-section of a wire 100 without coating 30 is lost. Moreover, by means of a coating 30, the outer surface of the wire 100 can be smoothed, e.g. resulting in a circular cross-section of the wire 100 without unevenness, so that foreign material will less likely deposit on the wire surface contaminating the wire 100. In addition, for iterative uses e.g. in endoscopic applications, the cleaning of the wire 100 becomes easier and more efficient.

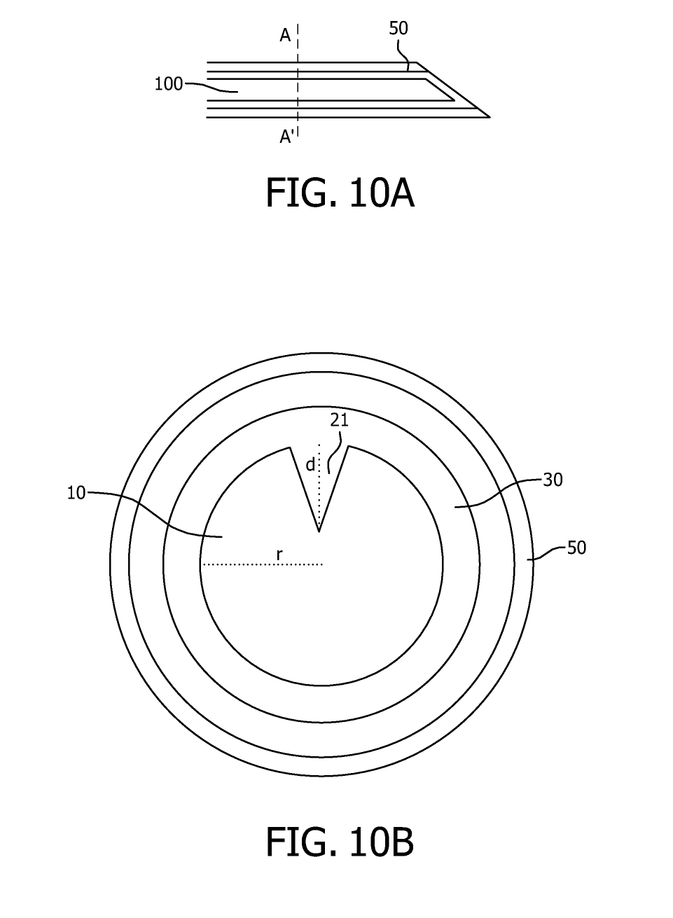

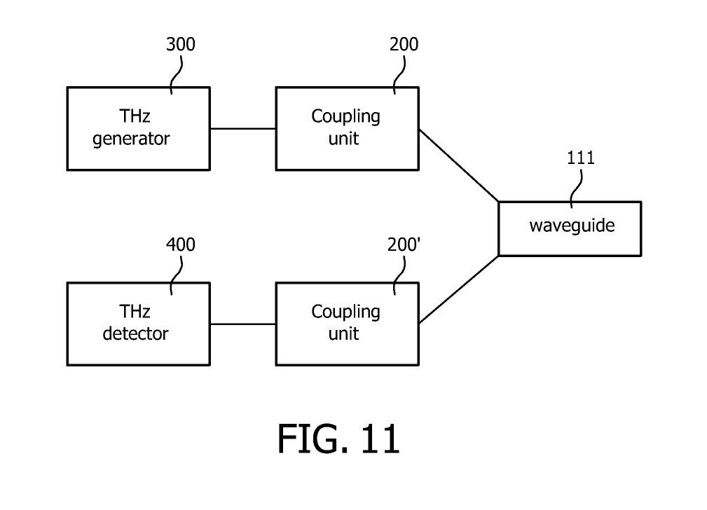

The wire 100 according to the present invention can be used in devices for a plurality of applications. For instance, the wire 100 may be included in a device for medical applications. In this case, the device comprising the wire 100 according to any above described embodiment can further include a catheter 50 or a medical needle. The flexibility of the wire-type waveguide according to the present invention, its low losses and low bending losses is particularly suitable for a terahertz endoscope. For endoscopic applications, the wire 100 may be arranged in a central hole of the catheter 50 in order to be introduced inside the human body, as shown in FIG. 10A. In order to focus the exiting electromagnetic waves to the area of interest, a tip of the wave-guiding wire 100 may be tapered or pointed. In addition, the device may include an output director, e.g. a mirror on the front end of the catheter in order to direct the electromagnetic signals towards a side surface of a cavity inside the human body. Due to the small diameter of the terahertz wave-guide according to the present invention and the strong confinement of the guided mode near the confinement structure, it is possible to place the wire 100 inside the catheter 50 and thereby guide the terahertz waves to the catheter tip, where the catheter 50 has an opening. At this place, the terahertz signals interact with the tissue and are partially reflected back into the wire 100. Then, the spectrum of the reflected signals can be measured to determine the nature of the tissue under observation. In FIG. 10B, a cross-section of the device shown in FIG. 10A along line A-A′ is illustrated. The inner diameter of the catheter 50 is larger than the outer diameter of the wire 100, which includes the coating 30, the groove-type confinement structure 21 and the core-structure 10.

In a further embodiment of the present invention, the wire 100 is employed in a spectroscopic or imaging system. For this, a wave-guiding device 111 includes the wire 100 according to one of the above-described embodiments. The wave-guiding device 111 can be connected via a coupling unit 200 to a terahertz generator 300 such that electromagnetic waves generated by the terahertz generator 300 can be coupled into the wire 100. Electromagnetic signals reflected back from the examination area can be coupled via the same coupling unit 200 to a terahertz detector 400. Alternatively, a second coupling unit 200′ may be provided for coupling the wave-guiding device 111 to the terahertz detector 400. After signal detection, the signals are analyzed using a signal processor and the like. Of course, the system may include further components of common spectroscopic systems, e.g. a memory, a display unit and the like. By these means, a localized terahertz spectrum can be provided at a specific location distant from the terahertz generator 300. The wave-guiding device 111 according to the present invention including a wire 100 as described above, can be used for general purposes for low-loss wave-guiding of high frequency signals, i.e. signals from below 100 GHz to several terahertz.

In one application, such a system could be applied in medical surgery for tissue analysis. Then, the wave-guiding device 111 can be a medical intervention device including a medical needle or a catheter 50, in which the terahertz wave-guiding wire 100 is integrated, as shown in FIG. 10. However, the described wire-type waveguide can also be applied to current terahertz time-domain spectrometers in order to guide terahertz signals and focus them to sub-wavelength dimensions. In this scenario, the terahertz signal is coupled into the wire 100 at its beginning and the ending tip of the wire 100 can be used to scan a sample. Terahertz radiation can be emitted and collected by the ending tip using common time-domain multiplexing techniques or by using two independent channels formed by confinement structures of the wire 100 as described above. Since the signal is focused to a sub-wavelength dimension, the image of the investigated surface will have a higher resolution than obtained with imaging systems based on free-propagating terahertz beams, wherein the resolution is limited by the wavelength of the used radiation, i.e. here in the order of millimeters. A still further application for the wire 100 and the wire-type wave-guiding device 111 according to the present invention lies in the domain of high-frequency measurements of integrated circuits using probe stations. Nowadays, measurements above 67 GHz by means of integrated circuits are highly unfeasible and must be carried out in bands due to the lack of coaxial cables that are able to work properly above 67 GHz. The use of the proposed wire-type waveguide may be a suitable and simple replacement for already existing rectangular waveguides and coaxial cables above the cut-off frequency.

According to the present invention, confinement and propagation of terahertz waves along a longitudinal direction of a wave-guiding wire can be achieved over long distances of several meters without substantial losses. The confinement of terahertz radiation to propagation in one dimension can be achieved by means of a wire with bounded cross-section having at least one positive and/or negative confinement structure, i.e. a rib or a groove. The advantage of such dimension-limited waveguides lies in their potential applications as well as in the appearance of different wave phenomena compared to planar waveguides. Thus, a high-frequency waveguide is proposed that is adapted to propagate high-frequency and wide-band signals from below 100 GHz to several terahertz. Moreover, since the wire-type waveguide is flexible, it has multiple application areas and is very versatile.

Furthermore, the wire-type waveguide according to the present invention can be manufactured by using common conductor materials such as copper. Thus, production costs should be comparable to those of regular copper wires.

1. (Currently Amended) A device for guiding electromagnetic waves in the terahertz range, comprising:
a wire (100) having a core structure (10) and at least one confinement structure (21, 22),
wherein the confinement structure (21, 22) extends continuously along a length of the wire (100) and includes at least one groove (21) formed in the core structure (10) and/or at least one rib (22) formed along the wire (100) prominent from the core structure (10).

2. The device according to claim 1, wherein the core structure (10) and the at least one confinement structure (21, 22) of the wire (100) are integrally formed and/or wherein the core structure (10) and the at least one confinement structure (21, 22) of the wire (100) are made of the same material and/or wherein the wire (100) is a profiled wire.

3. The device according to claim 1, wherein the core structure (10) has a substantially circular cross-section and/or the confinement structure (21, 22) has a substantially triangular and/or rectangular cross-section.

4. The device according to any one of the preceding claims, wherein at least one dimension of the confinement structure (21, 22) has sub-wavelength dimension and/or wherein dimensions of the confinement structure (21, 22) are smaller than the diameter of the core structure (10).

5. The device according to claim 1, wherein the core structure (10) and/or the confinement structure (21, 22) is made of a conducting material, a semiconductor material, a doped semiconductor material, a metal, copper and/or stainless steel.

6. The device according to claim 1, wherein the electromagnetic waves guided along the wire (100) comprise at least one propagation mode substantially confined within the confinement structure (21, 22) and/or within a cross-section of the wire (100).

7. The device according to claim 1, wherein the wire (100) is flexible.

8. The device according to claim 1, wherein the wire (100) comprises a coating (30).

9. The device according to claim 8, wherein the coating (30) surrounds the core structure (10) and the confinement structure (21, 22) such that the cross-section of the wire (100) has a circular, triangular or rectangular shape.

10. The device according to claim 8, wherein the coating (30) is a low-loss coating and/or wherein a material of the coating (30) comprises a metal, Benzocyclobutene and/or polyethylene.

11. The device according to claim 1, wherein the wire (100) comprises at least one pair of confinement structures (21, 22) facing each other that are coupled to each other.

12. The device according to claim 1, wherein the wire (100) comprises at least two confinement structures (21, 22) that are adapted to function as a transmitting and a receiving channel, respectively.

13. The device according to claim 1, wherein the device comprises further a needle (50) and/or a catheter for medical interventions, wherein a central hole is arranged in the needle (50) and/or the catheter with a larger diameter than a diameter of the wire.

14. A system for terahertz imaging, comprising:
a terahertz signal generator (300);
a terahertz signal detector (400); and
a device according to one of the preceding claims.

15. A method for guiding electromagnetic waves in the terahertz range, comprising the steps of:
coupling of electromagnetic waves into a wire (100) having a core structure (10) and at least one confinement structure (21, 22) including at least one groove (21) formed in the core structure (10) and/or at least one rib (22) formed along the wire prominent from the core structure (10) and extending continuously along a length of the wire (100); and
guiding the electromagnetic waves along the wire (100) to an area of interest.
